(19) United States
(12) Patent Application Publication
(10) Pub. No.: US 2026/0124339 A1
(43) Pub. Date: May 7, 2026

(54) DEPOSITION OF PHARMACEUTICAL INGREDIENT USING CONFINED ORGANIC PRINTING

(71) Applicant: Universal Display Corporation, Ewing, NJ (US)

(72) Inventors: Gregory McGraw, Yardley, PA (US); William T. Mayweather, III, Robbinsville, NJ (US); Ving Jick Lee, Wilmington, DE (US); Michael Hack, Carmel, CA (US)

(21) Appl. No.: 19/375,520

(22) Filed: Oct. 31, 2025

Related U.S. Application Data
(60) Provisional application No. 63/715,210, filed on Nov. 1, 2024.
(60) Provisional application No. 63/733,171, filed on Dec. 12, 2024.

(51) Int. Cl. A61L 27/28

(52) U.S. Cl. CPC A61L 27/28; A61L 2420/02; A61L 2420/08

(57) ABSTRACT

Embodiments of the disclosed subject matter provide a method of coating a surface with an active pharmaceutical ingredient, where an organic source having a first organic material may be heated to create an organic vapor, the first organic material having an active pharmaceutical ingredient. The source vapor may be transported via an inert carrier gas to a confined organic printing COP depositor in a deposition chamber. The organic vapor entrained in the inert carrier gas may be ejected from the COP depositor toward a surface to cause the first organic material to condense on the surface.

### FIELD

The present invention relates to devices and techniques for fabricating organic thin films for pharmaceuticals and prosthetics, and devices and techniques including the same.

### BACKGROUND

Presently, thin films can provide a delivery mechanisms for drugs, which are also referred to as active pharmaceutical ingredients (APIs). Such thin films can provide controlled release after ingestion, or the thin films can be administered through transdermal, mucosal, or ocular routes. Thin film APIs are typically applied to a surface using solvent based techniques like solvent casting, flexographic printing, and inkjet printing, which can be followed by a controlled thermal or freeze-drying process.

### SUMMARY

According to an embodiment, a method of coating a surface with an active pharmaceutical ingredient may be provided. The method may include heating an organic source comprising a first organic material to create an organic vapor, the first organic material comprising an active pharmaceutical ingredient. The source vapor may be transported via an inert carrier gas to a confined organic printing (COP) depositor in a deposition chamber. The organic vapor entrained in the inert carrier gas may be ejected from the COP depositor toward a surface to cause the first organic material to condense on the surface.

In the method, the organic source may be heated to a temperature of at least 150 C.

In the method, at least a portion of the COP depositor proximal to the surface may be maintained at a temperature of at least 150 C during ejection of the organic vapor toward the surface. The surface may have a temperature of 15-30 C during ejection of the organic vapor toward the surface.

A thickness of the organic material deposited on the surface may be 1-200 nm.

The surface may have an outer surface that has a plurality of granules. The method may include agitating the granules during at least a portion of time that the organic vapor entrained in the inert carrier gas is ejected toward the surface.

The surface may have a critical dimension of not more than 1.0 mm.

The surface may comprise at least a portion of a surface of an implantable device. The first organic material may be deposited on a first portion of the surface of the implantable device, and the method may include depositing a second organic material on a second portion of the surface of the implantable device via a COP depositor. The second portion may not overlap the first portion. The ejecting the organic vapor entrained in the inert carrier gas toward the surface may result in a deposition pattern that exceeds an intended deposition zone by not more than about 100 μm.

The surface may be a biocompatible material. The method may include depositing a second organic material on the first organic material deposited on the biocompatible material to form an ingestible tablet. The method may include selecting the amount of the first organic material, the second organic material, or a combination thereof based upon one or more attributes of a patient. The first and second organic materials may be deposited in alternating layers, a physical pattern on the biocompatible surface, or a combination thereof. The method may include depositing a third organic material on the second organic material.

The method may include measuring a fly height of the COP depositor above the biocompatible material while ejecting the organic vapor toward the surface, and may include adjusting the fly height of the COP depositor above the biocompatible material. The method may include adjusting the fly height to maintain a constant distance between an edge of the COP depositor and a closest surface of the surface and any materials deposited thereon.

The method may include maintaining a temperature gradient along a runline between the organic evaporation source and the COP depositor.

The method may include maintaining the deposition chamber at a pressure of [10-1000 Torr]

The method may include applying a confinement flow around the COP depositor, the surface, or a combination thereof.

The organic material deposited on the surface may not react chemically with any other material on the surface.

According to an embodiment, a system may be provided for depositing an active pharmaceutical ingredient on a surface. The system may include a crucible storing an organic material comprising an active pharmaceutical ingredient. A first controllable heater may be configured to heat the crucible and the organic material to a temperature of about 150-200 C. A runline may be configured to transport evaporated organic material from the crucible to a confined organic printing (COP) depositor. A second controllable heater may be configured to heat the runline to a temperature of about 150-200 C. A deposition chamber may include a target holder, the target holder configured to maintain a target comprising a deposition surface at a temperature of about 15-30 C. The COP depositor may be configured to eject the organic material toward the deposition surface of the target.

### BRIEF DESCRIPTION OF THE DRAWINGS

FIG. 1 shows a reactor for gas assisted deposition of active pharmaceutical ingredients (API) in the form of an organic thin film on a bed of granules according to an embodiment of the disclosed subject matter.

FIG. 2 shows a continuous reactor for the gas assisted deposition of API in the form of an organic thin film on a bed of granules according to an embodiment of the disclosed subject matter.

FIG. 3 shows a gas assisted deposition reactor for granule coating with multiple organic vapor sources according to an embodiment of the disclosed subject matter.

FIG. 4 shows a confined organic printing system for depositing thin film APIs on prosthetic implants according to an embodiment of the disclosed subject matter.

FIG. 5 shows an example of a dental implant with different types of coatings applied to different sections according to an embodiment of the disclosed subject matter.

FIG. 6 shows an example of a confined organic printing system combined with other pharmaceutical manufacturing operations in a common enclosure according to an embodiment of the disclosed subject matter.

FIG. 7 shows an example of a thin film stack structure in a tablet with a plurality of medicines that may be custom printed to meet the dosing needs of an individual patient according to an embodiment of the disclosed subject matter.

FIG. 8 shows an organic vapor jet printing tool to print tablets that are customized for an individual patient according to an embodiment of the disclosed subject matter.

### DETAILED DESCRIPTION

Unless otherwise specified, any of the layers of the various embodiments may be deposited by any suitable method. Preferred methods may include thermal evaporation, ink-jet, such as described in U.S. Pat. Nos. 6,013,982 and 6,087,196, which are incorporated by reference in their entireties, organic vapor phase deposition (OVPD), such as described in U.S. Pat. No. 6,337,102 to Forrest et al., which is incorporated by reference in its entirety, and deposition by organic vapor jet printing (OVJP), such as described in U.S. Pat. No. 7,431,968, which is incorporated by reference in its entirety. Other suitable deposition methods include spin coating and other solution-based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink-jet and OVJD. Other methods may also be used. The materials to be deposited may be modified to make them compatible with a particular deposition method.

Physical vapor deposition (PVD) of organic thin films onto biocompatible surfaces may facilitate the delivery of active pharmaceutical ingredients (APIs) in a variety of medical applications. The effectiveness and/or versatility of PVD in many of these applications may be enhanced by transporting organic vapor from evaporation sources to a deposition surface using an inert carrier gas. Embodiments of reactors and methods for coating granules with thin films of small molecule APIs using carrier gas assisted PVD are disclosed herein. Confined organic printing (COP) and organic vapor jet printing (OVJP) may be related gas assisted PVD techniques developed for OLED display manufacture. These techniques may be adapted to provide bioactive thin film coatings for pharmaceuticals, pharmaceutical products, and/or prosthetics are disclosed throughout.

As used herein, “top” means furthest away from a deposition surface, while “bottom” means closest to the deposition surface. Where a first layer is described as “disposed over” a second layer, the first layer is disposed further away from the surface. There may be other layers between the first and second layer, unless it is specified that the first layer is “in contact with” the second layer.

As used herein, “solution processible” means capable of being dissolved, dispersed, or transported in and/or deposited from a liquid medium, either in solution or suspension form.

Thin films are a topic of current research in the development of better delivery mechanisms for drugs, which are also referred to as active pharmaceutical ingredients (APIs). Thin films of APIs may be applied to granules of inert material for controlled release after ingestion. Thin film formulations of APIs may be administered through transdermal, mucosal, and/or ocular routes. Thin films of APIs may be applied to implantable structures like subdermal implants used for sustained delivery. Thin films of APIs may be used to apply surface treatments to implantable protheses like vascular stents. Thin film APIs may be applied using solvent based techniques like solvent casting, flexographic printing, and/or inkjet printing. These solvent based techniques may be followed by a controlled thermal process or a freeze-drying process.

Physical vapor deposition (PVD) may provide advantages over solvent based methods for depositing thin film coatings in pharmaceutical and prosthetic applications. Long drying times are not needed, and the danger of product contamination with a potentially toxic solvent is eliminated. Variation in dosage due to a “coffee ring” effect and/or other manifestations of surface tenson are also eliminated. The film thickness of material on the surface may be controlled with molecular monolayer precision. Small molecule APIs with molecular weight of less than 2000 g/mol may be particularly well suited for PVD. An advantage of PVD is that it may produce films with a very high loadings (100% concentration) of API compared to solvent processed films that may require API to be dispersed in a polymer matrix. This is advantageous for reducing the size of both pills and implantable pharmaceuticals.

Multicomponent coatings may be achieved by co-deposition of material from multiple vapor sources. A uniform, metastable glassy film may be deposited from a material mixture that does not form a thermodynamically stable solid solution. Such films can be readily grown using PVD and present unique advantages. For example, an API with poor water solubility may be co-deposited a water-soluble matrix. The PVD grown film may dissolve uniformly, promoting dispersal of the API, while a solvent deposited film is likely to leave behind a poorly soluble, API-rich crystalline phase as it dissolves.

Multilayer films may be grown by PVD. Molecularly sharp transitions in composition may be achieved between sequentially deposited layers in a thin film stack. This may not be possible with solvent based processing, since a solvent used in the deposition of one layer may dissolve material in a previous layer. Time-release drug delivery is a potential application for this capability. An API and an encapsulant may be dispensed on granules in alternating layers of a predetermined thickness. The encapsulant may dissolve at a known rate, liberating the API at desired intervals.

Graded concentration with film thickness may be achievable with PVD. The concentration of API in a layer of thin film may be varied with regards to thickness by adjusting the composition of API and binder material in the vapor phase as deposition progresses. For example, the concentration of API may be decreased as a film growth progresses so that the deeper portions of a film have a greater concentration of API. This may offset the reduction in surface area as the thin film dissolves so that the rate of API release may be uniform over time. Any combination of mixed, multilayer, and/or graded films may be deposited by PVD, which may help to solve outstanding issues in API delivery. The ability to deposit complex thin film stacks to aid in providing simultaneous time-release of multiple APIs remains area of active research.

Physical vapor deposition may permit the growth of film morphologies that may increase the bioavailability for APIs. Presently, thin films of antibiotic compounds can be deposited by vacuum thermal evaporation (VTE) to form high surface area dendritic films. These films have greater antimicrobial activity when applied to bacterial cell cultures than films applied using solvent. Other present arrangements have demonstrated that that high surface area dendritic films of the chemotherapy agent tamoxifen may be grown by organic vapor jet printing (OVJP). These films were found to be more effective at suppressing cell growth than powdered tamoxifen when applied to cultures of tumor cells.

Organic vapor jet printing and related carrier-gas assisted deposition methods may offer advantages beyond those of other PVD techniques in pharmaceutical manufacturing. OVJP may allow conformal deposition of organic thin films on uneven substrate or surface topography. Prior arrangements demonstrated this by coating an array of microneedles with a film of fluorescein. Confined organic printing (COP) is a gas-assisted thin film deposition technique generally intended for large area coating. Conversely, VTE is a line-of-sight technique, so prominent features on a substrate or surface tend to shadow underlying features, leading to uneven deposition. Techniques like Delivery-Exhaust-Confinement (DEC) OVJP and COP may not require a vacuum environment, unlike VTE. DEC OVJP and COP may not require a dedicated pressure vessel and are compatible with vacuum and pneumatic based substrate or surface handling techniques. DEC OVJP and COP may be combined with wet processing steps in the same environment. Patterned thin films applied by VTE require the use of a shadow mask. Direct patterning may be achieved by OVJP and related techniques. In one application, material printed by OVJP may be used to guide the placement and orientation of cells in a tissue culture. The ability of organic vapor jet printing to directly print patterns of a plurality of materials onto surfaces with areas, thicknesses, and/or layer structures controlled individually customizable print recipes may enable bespoke, time released formulations of medication to be dispensed for individual patients.

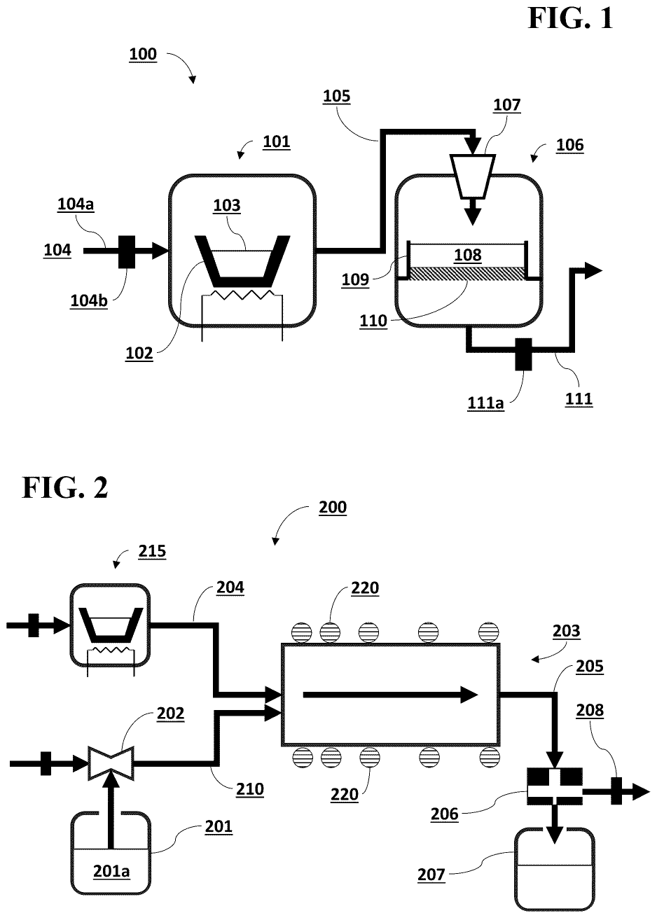

FIG. 1 shows a particle coating system 100 that uses organic vapor jet printing technology according to an embodiment of the disclosed subject matter. A sublimation source 101 may include an electrically heated crucible 102 with a charge of condensed organic material 103. The organic material 103 may evaporate or sublimate to form a vapor that is entrained in a stream of inert carrier gas 104 that passes through the sublimation source. The rate of gas flow (e.g., carrier gas 104) through the system 100 may be governed by a mass flow controller 104b on line 104a. The organic vapor and carrier gas mixture (i.e., a mixture of carrier gas 104 and organic vapor that is formed from the organic material 103) may pass through a heated runline 105 to a deposition chamber 106. The runline 105 may heated to prevent condensation of the organic vapor. The organic vapor may be dispensed into the deposition chamber 106 through a heated nozzle assembly 107. The nozzle assembly 107 may have the same construction as an OVJP nozzle, which causes the carrier gas and entrained material(s) to be ejected from the nozzle assembly in a collimated jet. Alternatively, the nozzle assembly 107 may use a COP print head to eject a diffuse stream of organic vapor entrained in carrier gas. A COP-type print head may be preferred for coating granules or other particles to improve coverage of many particles at once. The granules 108 to be coated may be loaded into a tray 109 in the deposition chamber 106. The granules 108 may be supported in the tray 109 by a fine sieve or porous frit 110 that may permit gas flow. The edges of the tray 109 may be flush with an inner surface of the deposition chamber 106 so gas flow is guided through a particle bed. Gas may be extracted from the deposition chamber 106 by an exhaust line 111 on the downstream side of the tray. The pressure in the deposition chamber 106 may be governed by a pressure controller 111a on the exhaust line 111.

The path of gas flow from the heated nozzle assembly 107 through the deposition chamber 106 may be configured to maximize contact between the organic vapor and granule bed, while minimizing contact between the organic vapor and the walls of the deposition chamber 106. The deposition chamber 106 may be cold-walled. The tray 109 may include an agitator to improve the uniformity of coating on the granules 108. The tray 109 may also have a temperature controller, a heater, and/or a cooling device to control the temperature of deposition. The particle coating system 100 may be operated in batch mode, with the granules 108 loaded and unloaded between deposition operations.

In some embodiments, the particle coating system 100 may include a device to continuously load fresh granules 108 and unload coated particles while the system is in operation, as shown in system 200 of FIG. 2. Granules 201a stored in a hopper 201 may be entrained in an inert gas stream using a venturi 202. The granule bearing gas jet 210 is then introduced into the downstream end of a hot walled reactor 203. The granule bearing gas jet may be mixed with a jet of organic vapor from an evaporation source 215 and heated runline 204, similar to the runlines and flow controllers shown in FIG. 1 and described above. The reactor 203 may be configured to mix the streams of granule and organic vapor bearing gasses and to prevent aerosolized particles from settling out of the flow. Organic material may condense on the particles as the mixture moves downstream in the reactor 203. The reactor 203 may have a temperature gradient such that temperature decreases in a downstream direction to promote condensation. For example, a series of heaters 220 arranged around or integrated with one or more walls of the reactor chamber 203 may be used to heat the chamber to a desired temperature range and gradient. Aerosolized particles may be removed from the effluent stream 205 that leaves the reactor 203 using a virtual impactor 206 and may be deposited in a hopper 207. The remaining process gas may be exhausted through a pressure controller 208 that governs reactor pressure. Other designs like a fluidized bed reactor may be used to bring continuous streams of organic vapor and fresh granules into contact.

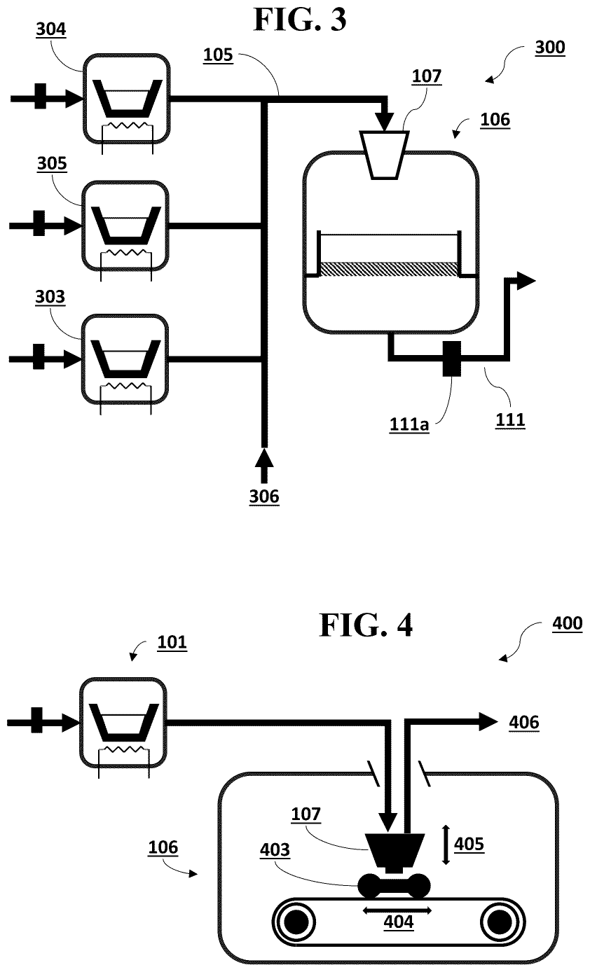

Granules may be coated with a mixture of organic compounds using the deposition system 300 shown in FIG. 3, which functions the same as the system 100 shown in FIG. 1 other than the addition of multiple sources as shown. A plurality of organic vapor source assemblies may be arranged along a heated runline 105 leading to a deposition chamber 302. Sources may be arranged so that the source 303 with the lowest operating temperature, usually containing the most volatile component, is at the furthest upstream position on the runline 105. The source 304 with the highest operating temperature, usually containing the least volatile component, is in the furthest downstream position on the runline 105 before the deposition chamber 106. Remaining materials may be arranged in sources 305 so that each source has a higher operating temperature than the source upstream of it and a lower operating temperature than the source downstream of it. Each source may be attached to a corresponding inlet with mass flow controller as previously disclosed with respect to FIG. 1, or equivalent arrangement that allows for control of the flow of material entering the source. A temperature gradient may be maintained along the runline 105 such that temperature increases monotonically in the downstream direction. A flow of inert gas 306 may be introduced to the far upstream portion of the runline 105 for the deposition process.

The molar rate \dot{n} at which each material may be evolved by an organic vapor source governed by both the temperature of the source, which sets the vapor pressure P₀ inside the source, and the volumetric flow rate \dot{V} of carrier gas through the source, such that \dot{n}=P₀\dot{V}/(RT) where T is the source temperature and R is the ideal gas constant. Overall deposition rate and the ratios of the thin film components may be controlled by balancing the temperatures and flow rates through the array of material sources.

Macroscopic items may be coated using confined organic printing to coat swathes on the item surface. System 400 shown in FIG. 4 may include one or more evaporation sources 101 as previously disclosed, configured to generate a stream of organic vapor laden inert gas that is transmitted to an organic vapor deposition head 107, which may be a COP-type depositor that emits a relatively wide, diffuse stream of gas to allow for uniform coating of granules. In other arrangements, the deposition head 107 may be an OVJP-type print head that allows for printing small, precise patterns on the surface of the device 403. The evaporation source(s) 101 may include a set of sources 303, 304, 305, as shown in FIG. 3. Material may be deposited on desired regions of the surface of a workpiece such as a prosthetic implant or other implantable device 403. The workpiece may be moved parallel to the plane of the deposition surface by a web or stage, as shown by arrows 404. The deposition head 107 may be moved normal to the deposition surface 403 as shown by the arrows 405, to maintain a constant fly height above the part (e.g., prosthetic implant 403) using a sensor and actuator system that responds to the topography of the part to maintain a constant fly height above its surface. Excess organic vapor may be taken up by an exhaust flow 406 drawn from the gas surrounding the deposition head 107. The deposition chamber 106 may be filled with an inert gas atmosphere at a specified pressure for the process. Both COP and the particle coating reactor described above in connection with FIG. 1 may be adapted for either fiber coating or roll-to-roll coating of a biocompatible polymer surface.

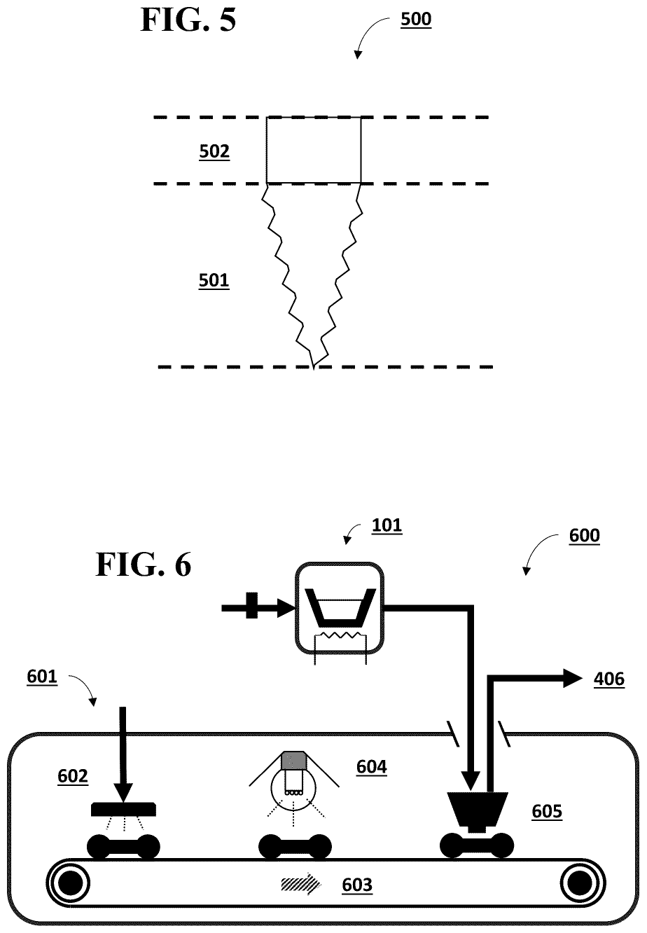

Confined organic printing may have a relatively low-resolution maskless patterning capability, permitting it to coat features with a critical dimension of 1 mm or less. An example of the use of confined organic printing is shown in FIG. 5, in which an example dental implant 500 includes a screw portion 501 in a first zone and a crown 502 in a second zone, each with a separate thin film coating. External threads of a screw portion 501 may coated with a mixture of APIs to promote fusion with bone. An upper portion that accepts a crown 502 may be coated with APIs that promote healing with the gum. Delivery Exhaust Confinement (DEC) OVJP may be used to pattern API films with finer features as needed.

Confined organic printing, DEC OVJP, and related techniques may capture surplus organic vapor and do not contaminate the far field. This is an additional advantage over VTE. Confined organic printing, DEC OVJP, and related techniques may be performed within the same environment as other operations in a manufacturing process. An example process 600 is shown in FIG. 6. Three operations may be performed within a common enclosure 601, which may be a nitrogen glovebox (i.e., nitrogen chamber) or the like. The part may be spray coated in a UV curable resin 602, and then may be transferred on a web 603 to a UV curing lamp 604. The part may be transferred to an organic vapor deposition head 605 for an organic thin film deposition operation.

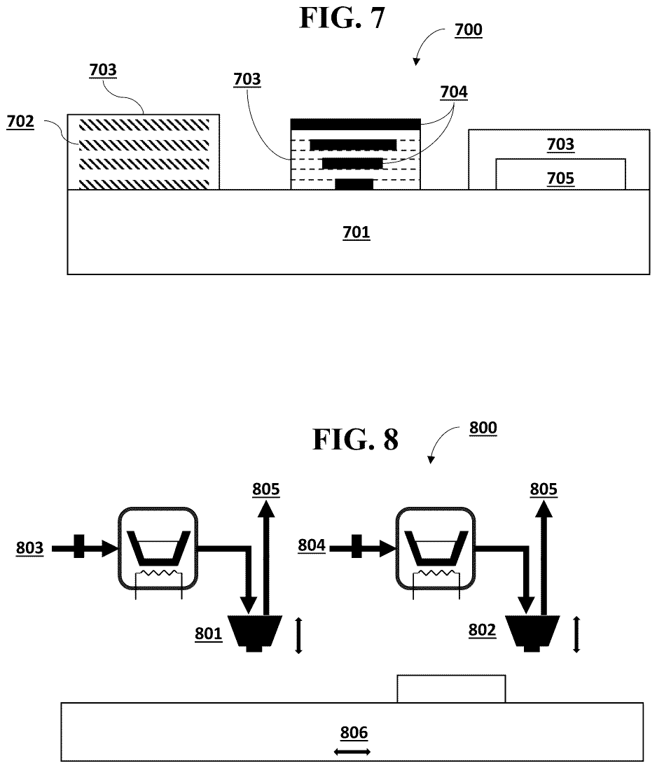

FIG. 7 shows a schematic example of a custom fabricated tablet 700 for oral drug delivery for an individual patient. The APIs may be printed by OVJP on a surface 701 of starch, synthetic polymer, or any other material known in the art. A first API 702 (i.e., drug A) may be deposited in an alternating thin film stack with an encapsulant 703 that slows its dissolution. This ensures that the patient may receive regular doses of drug A throughout the day. A second API 704 (i.e., drug B) may be printed in alternating layers with an encapsulant, but the width of the stripes of the drug B film vary so that wider swathes of film are located near the top of the stack, while narrower stripes are located near the surface 701. The release rate of drug B may therefore decrease as the film stack is dissolved. If the drug B and its encapsulant dissolve at a known rate, the dosage delivered throughout the day may be controlled in this manner. A third API 705 (i.e., drug C) may deposited on the surface and encased in the encapsulant. Drug C may be configured to release when a predetermined interval after ingestion has occurred that may be based on the thickness of the encapsulant. This may be a method of providing multiple drugs needed at different times of the day in a single dose. By controlling the areas of the tablet printed with API and the thicknesses of encapsulant, an entire schedule of dosages for multiple medications may be tailored to the needs of a single patient. In an embodiment, API 702, API 704, and API 705 may be the same drug but in different concentrations, for control of the overall dosage over duration of the tablet printed. The top and bottom of each deposited film of the second API 704 and encapsulant 703 within the stack is shown with dotted lines to aid in visualization, but it will be understood that the thicknesses and widths of each layer may vary as disclosed, and any desired arrangement of drug films may be used.

In an embodiment, another approach to change the release rate of an API over time to tailor drug delivery to achieve a predetermined dosage over time is to deposit the API in an inert and benign host material so that the API and host material can be deposited using OVJP and mixed. Such a mixture allows for the mixture of host and API materials to be controlled in much the same way that mixtures of dopants and hosts are controlled in OLED deposition, such as by using mass flow controllers to adjust the rate of one material flow relative to the other. The flow rates also may be adjusted over time so there is a concentration profile of API in the deposited film as a function of film thickness, which in turn results in the drug release varying over time as the film stack is dissolved. In other words, by modifying the concentration profile of the API in the inert and benign host material, the final structure that is created may have varying and controllable concentrations of API throughout the final structure. Using this approach we can deposit API films with pre-determined concentration profiles to achieve specific drug release rates over time. Alternatively or in addition, multiple drugs may be mixed together in one portion of the film and their concentrations profiled, similar to having two dopants and one host in an OLED. These features also may be combined with those discussed above.

FIG. 8 shows an OVJP system 800 that may be used to fabricate the tablet shown in FIG. 7. The OVJP system 800 may include two OVJP deposition heads and their associated evaporation sources, one OVJP deposition head 801 may be loaded with an API and the other OVJP deposition head 802 may be loaded with an encapsulant intended to slow dissolution of the API. More sources may be attached to a head if a more complex mixture is needed. Both heads 801, 802 may be fed with inert carrier gas 803, 804 and may have exhaust lines 805 to remove surplus organic material. The print heads 801, 802 are the same type and structure as the COP- or OVJP-style print head 107 as previously described. The surface 701 of the tablet 700 shown in FIG. 7 may serve as the deposition surface and may sit on a motion stage 806 that translates it between heads 801, 802 and normal to the page as needed. Printing on the surface 701 may be initiated for each deposition head 801, 802 by moving head 801, 802 normal to the plane of the stage with an actuator to bring it into proximity with the surface 701. A sensor may be used to control fly height of deposition head 801, 802 through a feedback loop. A plurality of APIs may be deposited on the surface 701 by either having a plurality of deposition heads and/or by swapping the organic vapor sources attached to the deposition heads. The patterns, print speeds, and/or numbers of print passes of the stage may be digitally controlled (e.g., by a controller) for individual surfaces (e.g., surface 701 or the like) and may be adjusted as needed, permitting bespoke formulations of medicines to be printed. Because the printing process does not require vacuum and it does not contaminate the far-field, drugs may be dispensed on the surface 701 using solvent based techniques as well as OVJP.

In the embodiments of the disclosed subject matter shown in FIGS. 1-8 and described above, a method of coating a surface with an active pharmaceutical ingredient may be provided. The method may include heating (e.g., by electrically heated crucible 102) an organic source comprising a first organic material (e.g., condensed organic material 103) to create an organic vapor, the first organic material comprising an active pharmaceutical ingredient. The source vapor may be transported via an inert carrier gas (e.g., inert carrier gas 104; inert carrier gas 803, 804) to a confined organic printing COP depositor (e.g., nozzle assembly 107; heads 801, 802) in a deposition chamber (e.g., deposition chamber 106). The organic vapor entrained in the inert carrier gas may be ejected from the COP depositor toward a surface (e.g., surface 701) to cause the first organic material to condense on the surface.

In some embodiments, the organic source may be heated to a temperature of at least 150 C. In some embodiments, at least a portion of the COP depositor proximal to the surface may be maintained at a temperature of at least 150 C during ejection of the organic vapor toward the surface. The surface may have a temperature of 15-30 C during ejection of the organic vapor toward the surface.

A thickness of the organic material deposited on the surface may be 1-100 nm. The surface may have an outer surface that has a plurality of granules (e.g., granules 108). The outer surface having the granules may be formed by agitating the granules (e.g., by agitator in tray 109) during at least a portion of time that the organic vapor entrained in the inert carrier gas is ejected toward the surface. In some embodiments, the surface may have a critical dimension of not more than 1.0 mm.

In some embodiments, the surface may be at least a portion of an implantable device (e.g., prosthetic implant 403, dental implant 500, and the like). That is, the first organic material may be deposited on a first portion (e.g., screw portion 501) of the surface of the implantable device, and the a second organic material may be on a second portion (e.g., crown 502) of the surface of the implantable device via a COP depositor. In some embodiments, the second portion may not overlap the first portion.

In some embodiments, the ejecting the organic vapor entrained in the inert carrier gas toward the surface may result in a deposition pattern that exceeds an intended deposition zone by not more than about 100 μm. A mask may be used to form the deposition pattern, or the deposition pattern may be formed without a mask. In some embodiments, material wasted on overspray may be minimized. The 100 microns may refer to the maximum distance measured normal to the deposition surface, in a direction away from the surface, at which material is deposited, where spray outside of the maximum distance may be overspray.

The surface on which APIs are deposited in embodiments disclosed herein may be a biocompatible material. In some embodiments, a second organic material (e.g., second API 704) may be deposited on the first organic material (e.g., first API 702), where the first organic material is deposited on the biocompatible material (e.g., surface 701). The layering of the second organic material, the first organic material, and the biocompatible material may form an ingestible tablet (e.g., tablet 700). The amount of the first organic material, the second organic material, or a combination thereof may be based upon one or more attributes of a patient. The first and second organic materials may be deposited in alternating layers, a physical pattern on the biocompatible surface, or a combination thereof. In some embodiments, a third organic material (e.g., third API 705) may be deposited on the second organic material. In some embodiments, other thin films and/or self-assembled films may be deposited over the surface on which APIs are deposited, or between layers of APIs deposited on a common surface, such as granules or other surfaces 108, 403, 701, 806. For example, a material may be vapor deposited over uncoated granules 108 before an API is deposited to improve wetting of the API.

In some embodiments, a fly height of the COP depositor may be measured above the biocompatible material while ejecting the organic vapor toward the surface, and may include adjusting the fly height of the COP depositor above the biocompatible surface. A fly height of the COP depositor may be adjusted to maintain a constant distance between an edge of the COP depositor and a closest surface of the surface and any materials deposited thereon.

In some embodiments, a temperature gradient may be maintained along a runline between the organic evaporation source and the COP depositor.

The deposition chamber may be maintained at a pressure of 10-1000 Torr.

In some embodiments, a confinement flow may be applied around the COP depositor, the surface, or a combination thereof. In some embodiments, the organic material deposited on the surface may not react chemically with any other material on the surface.

A system according to embodiments of the disclosed subject matter may be provided for depositing an active pharmaceutical ingredient on a surface. The system may include a crucible (e.g., electrically heated crucible 102) storing an organic material (e.g., condensed organic material 103) comprising an active pharmaceutical ingredient. A first controllable heater may be configured to heat the crucible (e.g., electrically heated crucible 102) and the organic material to a temperature of about 150-200 C. A runline (e.g., runline 105) may be configured to transport evaporated organic material from the crucible to a confined organic printing (COP) depositor (e.g., heated nozzle assembly 107). A second controllable heater may be configured to heat the runline to a temperature of about 150-200 C. A deposition chamber may include a target holder (e.g., motion stage 806), the target holder configured to maintain a target comprising a deposition surface at a temperature of about 15-30 C. The COP depositor may be configured to eject the organic material toward the deposition surface of the target. The methods and devices disclosed herein may be used to fabricate a variety of devices, including ingestible items such as tablets and/or implantable device suitable for delivery of APIs to or within the body. For example, a device fabricated using the techniques disclosed herein may include a deposited film that has a concentration of one or more APIs that varies by thickness, such as the arrangements shown in FIG. 7. The APIs may be distributed within an inert host. As another example, a device fabricated as disclosed herein may include one or more APIs deposited across two or more pairs of layers or layered structures, such as those shown in FIG. 7 and described throughout the present disclosure. A pair of layers may include, for example, a first layer that includes the API(s), and a second layer that includes a host, which may be an inert host. The thicknesses of the layers may vary for some or all of the pairs of layers in the device.

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

We claim:

1. A method of coating a surface with an active pharmaceutical ingredient, the method comprising:
heating an organic source comprising a first organic material to create an organic vapor, the first organic material comprising an active pharmaceutical ingredient;
transporting the organic vapor via an inert carrier gas to a confined organic printing (COP) depositor in a deposition chamber;
ejecting the organic vapor entrained in the inert carrier gas from the COP depositor toward a surface to cause the first organic material to condense on the surface.

2. The method of claim 1, further comprising:
mixing the source vapor with an inert host material vapor; and
ejecting the inert host material vapor toward the surface to cause the first organic material to be mixed with the inert host material during deposition of the first organic material on the surface.

3. The method of claim 1, wherein the organic source is heated to a temperature of at least 150 C.

4. The method of claim 1, wherein at least a portion of the COP depositor proximal to the surface is maintained at a temperature of at least 150 C during ejection of the organic vapor toward the surface.

5. The method of claim 4, wherein the surface has a temperature of 15-30 C during ejection of the organic vapor toward the surface.

6. The method of claim 1, wherein a thickness of the organic material deposited on the surface is 1-200 nm.

7. The method of claim 1, wherein the surface comprises an outer surface of a plurality of granules.

8. The method of claim 7, further comprising agitating at least a portion of the plurality of granules during at least a portion of time that the organic vapor entrained in the inert carrier gas is ejected toward the surface.

9. The method of claim 1, wherein the surface comprises at least a portion of a surface of an implantable device.

10. The method of claim 9, wherein the surface has a critical dimension of not more than 1.0 mm.

11. The method of claim 9, wherein the first organic material is deposited on a first portion of the surface of the implantable device, the method further comprising:
depositing a second organic material on a second portion of the surface of the implantable device via a COP depositor.

12. The method of claim 11, wherein the second portion does not overlap the first portion.

13. The method of claim 9, wherein the ejecting the organic vapor entrained in the inert carrier gas toward the surface results in a deposition pattern that exceeds an intended deposition zone by not more than about 100 μm.

14. The method of claim 1, wherein the surface comprises a biocompatible material.

15. The method of claim 14, further comprising depositing a second organic material on the first organic material deposited on the biocompatible material.

16. The method of claim 15, wherein the first and second organic materials form an ingestible tablet.

17. The method of claim 15, wherein each of the first and second organic materials is mixed with a corresponding inert host while being deposited.

18. The method of claim 15, further comprising selecting the amount of the first organic material, the second organic material, or a combination thereof based upon one or more attributes of a patient.

19. The method of claim 15, wherein the first and second organic materials are deposited in alternating layers, in a physical pattern on the biocompatible material, or a combination thereof.

20. The method of claim 15, further comprising depositing a third organic material on the second organic material.

21. The method of claim 1, further comprising:
measuring a fly height of the COP depositor above the biocompatible material while ejecting the organic vapor toward the surface; and
adjusting the fly height of the COP depositor above the biocompatible material.

22. The method of claim 21, further comprising adjusting the fly height to maintain a constant distance between an edge of the COP depositor and a closest surface of the surface and any materials deposited thereon.

23. The method of claim 1, further comprising maintaining a temperature gradient along a runline between the organic evaporation source and the COP depositor.

24. The method of claim 1, further comprising maintaining the deposition chamber at a pressure of 10-1000 Torr.

25. The method of claim 1, further comprising applying a confinement flow around the COP depositor, the surface, or a combination thereof.

26. The method of claim 1, wherein the organic material deposited on the surface does not react chemically with any other material on the surface.

27. The method of claim 1, further comprising:
depositing a thin film or self-assembled film on the surface prior to ejecting the organic vapor entrained in the inert carrier gas from the COP depositor toward the surface.

28. A system for depositing an active pharmaceutical ingredient on a surface, the system comprising:
a crucible storing an organic material comprising an active pharmaceutical ingredient;
a first controllable heater configured to heat the crucible and the organic material to a temperature of about 150-200 C;
a runline configured to transport evaporated organic material from the crucible to a confined organic printing (COP) depositor;
a second controllable heater configured to heat the runline to a temperature of about 150-200 C;
a deposition chamber comprising a target holder, the target holder configured to maintain a target comprising a deposition surface at a temperature of about 15-30 C;
wherein the COP depositor is configured to eject the organic material toward the deposition surface of the target.

29. A device fabricated using the method of claim 2, wherein the first organic material comprises one or more active pharmaceutical ingredients and wherein the device comprises a deposited film that has a concentration of the one or more active pharmaceutical ingredients that varies by thickness.

30. The device of claim 29, wherein the host is an inert host.

31. A device fabricated using the method of claim 1, wherein the first organic material comprises one or more active pharmaceutical ingredients, and wherein the device comprises two or more pairs of layers, wherein a pair of layers comprises a first layer comprising the one or more active pharmaceutical ingredients and a second layer comprising a host.

32. The device of claim 31, wherein the host is an inert host.

33. The device of claim 31, wherein the thickness of the first layer relative to the thickness of the second layer varies for some or all of the pairs of layers of the two or more pairs of layers.
